(19) United States
(12) Patent Application Publication
(10) Pub. No.: US 2025/0127665 A1
(43) Pub. Date: Apr. 24, 2025

(54) closing element for producing absorbent sanitary articles

(71) Applicant: Fameccanica.Data S.p.A., San Giovanni Teatino Chieti (IT)

(72) Inventor: Gabriele SABLONE, San Giovanni Teatino Chieti (IT)

(21) Appl. No.: 18/921,129

(22) Filed: Oct. 21, 2024

(30) Foreign Application Priority Data
Oct. 23, 2023 (IT) 102023000022089

(51) Int. Cl. A61F 13/56

(52) U.S. Cl. CPC A61F 13/5638; A61F 2013/5677

(57) ABSTRACT

A closing element for producing reclosable absorbent sanitary articles includes a non-woven sheet folded according to a general omega-shaped configuration and including a base, two intermediate flaps connected to respective ends of the base by respective first folds, and two distal flaps connected to respective ends of the respective intermediate flaps by respective second folds, wherein the base has a dimension between 15 and 90 mm and each of the intermediate flaps has a dimension between 5 and 30 mm.

### FIELD OF THE INVENTION

The invention relates to a closing element intended to be used for producing absorbent sanitary articles.

The invention was developed with particular attention to producing absorbent sanitary articles that can be worn like pants, such as so-called training pants, according to the production technique known as “Cross Direction”.

### DESCRIPTION OF THE PRIOR ART

A well-established technique for producing wearable absorbent sanitary articles in the form of reclosable panty-type underwear consists of forming a continuous composite web movable in a machine direction, and formed of a continuous chain of article blanks arranged with their respective longitudinal axes transverse to the machine direction. This production technique called Cross Direction is different from the production technique called Machine Direction which involves producing the absorbent sanitary articles while they advance with their longitudinal axes parallel to the machine direction.

Examples of methods for producing absorbent sanitary articles according to the Cross Direction technique are described in documents EP-A-1013251, IT1379452, IT1410464 and IT1410465 by the same Applicant.

The continuous composite tape forming the chain of product blanks normally comprises two elastic bands parallel to the machine direction and spaced apart in a direction perpendicular to the machine direction, and a plurality of absorbent cores extending between the two elastic bands perpendicular to the machine direction. The continuous composite tape advancing in the machine direction is folded along a longitudinal axis parallel to the machine direction, so that the two elastic bands overlap each other.

For producing refastenable absorbent sanitary articles, refastenable closing elements are applied to the continuous composite tape, which are coupled with complementary closing elements after longitudinal folding of the tape.

EP-A-1523968 by the same applicant describes a closing element for producing absorbent sanitary articles wearable in the form of panties, comprising a non-woven sheet folded according to a general omega-shaped configuration and including a base subdivided into two sections located on opposite sides of a central plane, two intermediate flaps located on opposite sides of the central plane, connected to respective outer ends of the base sections and permanently fixed to the base, two distal flaps located on opposite sides of the central plane, connected to respective inner ends of the respective intermediate flaps by respective folds and fixed in a separable manner to respective intermediate flaps. Respective surface fasteners are fixed on surfaces of respective distal flaps opposite to the base. In one embodiment the lengths of the distal flaps are greater than the lengths of the intermediate flaps, so that the distal flaps protrude outwards from the outer ends of the base.

The base of the closing element is fixed to a waistband of a sanitary article. Since the base of the closing element is not elastic, the area of the elastic band on which the base of the closing element is fixed becomes inelastic and reduces the length of the elastic part of the waistband.

### OBJECT AND SUMMARY OF THE INVENTION

The object of the present invention is to provide a closing element with an optimized design so as to increase, all other conditions being equal, the length of the elastic part of the waistbands of the sanitary articles.

According to the present invention, this object is achieved by a closing element having the characteristics forming the subject of claim 1.

The claims form an integral part of the disclosure provided here in relation to the invention.

### BRIEF DESCRIPTION OF THE DRAWINGS

The present invention will now be described in detail with reference to the attached drawings, given purely by way of non-limiting example, in which:

FIG. 1 is a schematic perspective view of a particular embodiment of a closing element according to the present invention,

FIG. 2 is a schematic cross-section of the closing element of FIG. 1, and

FIG. 3 is a schematic perspective view of a particular embodiment of a closing element according to the present invention.

### DETAILED DESCRIPTION

With reference to the Figures, numeral 10 indicates a closing element for producing reclosable absorbent sanitary articles.

The closing elements 10 are intended to be fixed at spaced apart positions on a continuous elastic band in a process for producing absorbent sanitary articles according to the technique called Cross Direction, as described in EP-A-1523968 by the same applicant.

The closing element 10 comprises a non-woven sheet 12 folded according to a general omega-shaped configuration.

The folded non-woven sheet 12 comprises a generally flat base 14 subdivided into two sections 16 located on opposite sides of a central plane X perpendicular to the base 14.

The folded sheet non-woven 12 comprises two intermediate flaps 18 parallel to the base 14 and located on opposite sides of the central plane X. The intermediate flaps 18 are connected to respective ends of the base 14 by respective first folds 20. The intermediate flaps 18 are permanently fixed to the base 14, for example, by respective layers of glue 22 or, alternatively, by thermal or ultrasonic welding.

The folded non-woven sheet 12 comprises two distal flaps 24 parallel to the respective intermediate flaps 18 and located on opposite sides of the central plane X. The distal flaps 24 are connected to respective ends of the respective intermediate flaps 18 by respective second folds 26. The distal flaps 24 protrude laterally from the base 14 beyond the respective first folds 20.

The two distal flaps 24 are detachably fixed to the respective intermediate flaps 18 by temporary joining areas 28, which are susceptible to breaking when subjected to a light force so as to allow the mutually facing surfaces of the distal flaps 24 and the intermediate flaps 18 to separate. The temporary bonding areas 28 may be formed by thermal or ultrasonic welding or by technical glue dots.

The fastening element 10 comprises at least two surface fastening elements 30 fixed to surfaces of the distal flaps 24 facing away from the base 14. The surface fastening elements 30 may be micro-hook fastening elements designed to form a Velcro®-type surface closure with corresponding micro-loop fastening elements.

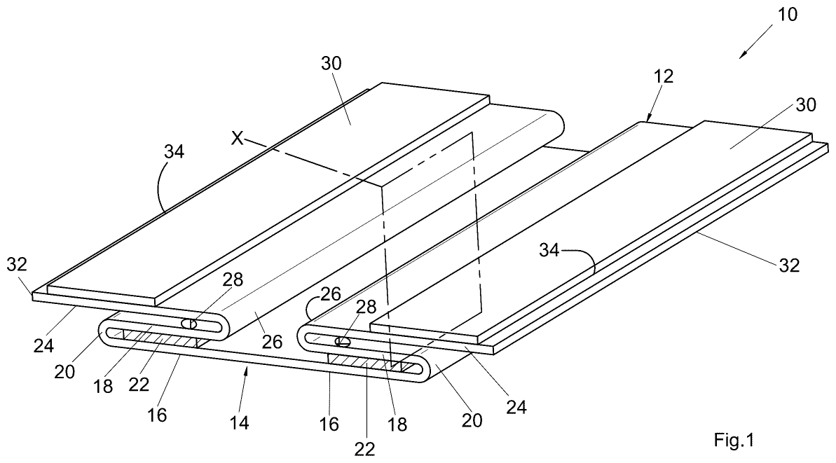

Referring to FIG. 1, for each distal flap 24 the surface fastening elements 30 may be continuous along a direction parallel to the central plane X.

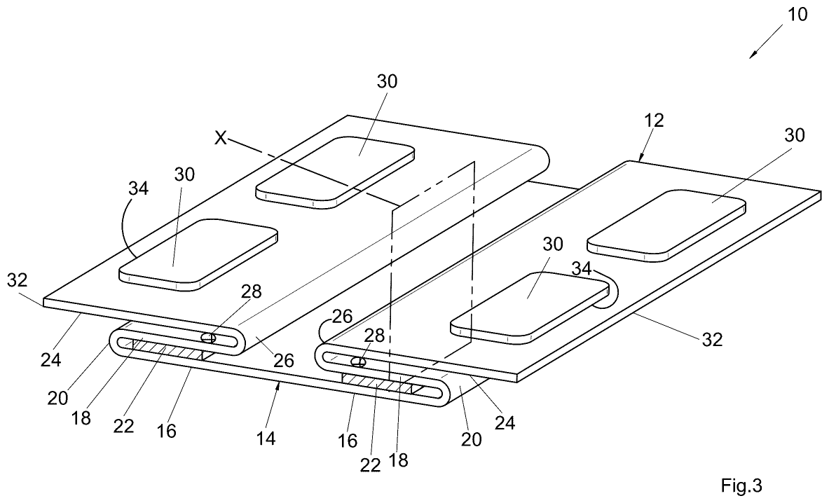

Referring to FIG. 3, for each distal flap 24 the surface fastening elements 30 may be discontinuous along a direction parallel to the central plane X.

In accordance with particular embodiments, the surface fastening elements 30 may be made entirely implemented on surfaces portions of the distal flaps 24 opposite to the base 14 by means of, purely by way of example and not limitative, an ultrasonic apparatus such as that described in the patent application EP4108220 by the same Applicant.

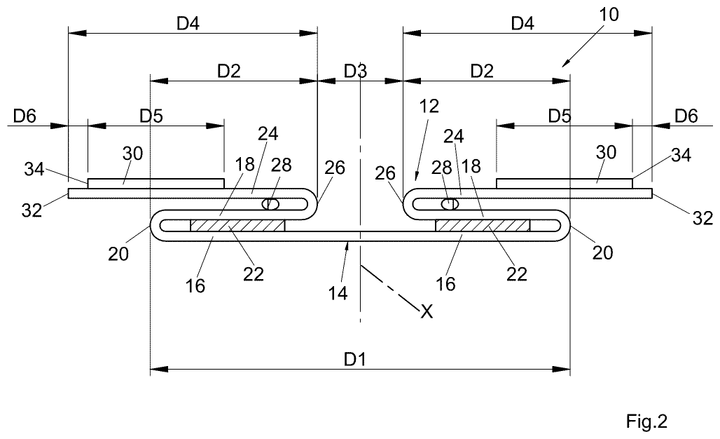

With reference to FIG. 2, the base 14 has a dimension D1 in a direction perpendicular to the central plane X equal to the distance between the first folds 20 and between 15 and 90 mm.

Each of the intermediate flaps 18 has a dimension D2 in a direction perpendicular to the central plane X equal to the distance between a first fold 20 and a second fold 26 and between 5 and 30 mm.

The dimension D3 in a direction perpendicular to the central plane X equal to the distance between the second folds 26 is between 5 and 30 mm.

The distance between each second fold 26 and the central plane X, equal to D3/2, is less than the dimension D2 of each intermediate flap 18, or rather: D3/2<D2.

Each distal flap 24 has a dimension D4 in a direction perpendicular to the central plane X equal to the distance between the second fold 26 and a respective end 32 of the distal flap 24 between 10 and 76 mm.

The dimension D4 of each distal flap 24 may be greater than the sum of the dimension D2 of each intermediate flap 18 and half the dimension D1 of the base 14, i.e. D4>D2+D1/2.

Each surface fixing element 30 may have a dimension D5 in a direction perpendicular to the central plane X of between 5 and 25 mm.

Each distal flap 24 has a dimension D6 in a direction perpendicular to the central plane X equal to the distance between the end 32 and the outer edge 34 of the corresponding surface fixing element 30 between 3 and 12 mm.

It is understood that for an expert in the field the dimensions D1, D2, D3, D4, D5 and D6 are easily measurable on a closing element 10 of an absorbent sanitary article in accordance with the present invention by suitable length measuring instruments, such as for example a millimeter tape or a caliper.

In particular, an expert in the field would be able to trace the dimension D3 by measuring on a closing element 10 of an absorbent sanitary article in accordance with the present invention the distance between each second fold 26 and the central plane X, equal to D3/2, and doubling its value.

Furthermore, it may be technically reasonable for an expert in the field to determine for the same dimension D1, D2, D3, D4, D5 and D6 a representative value of multiple measurements taken along a direction parallel to the central plane X, such as the arithmetic mean, and compare this representative value with the respective distance intervals D1, D2, D3, D4, D5 and D6 reported above.

Of course, without prejudice to the principle of the invention, the details of construction and the embodiments can be widely varied with respect to those described and illustrated, without thereby departing from the scope of the invention as defined by the claims that follow.

1. A closing element for producing reclosable absorbent sanitary articles, comprising a non-woven sheet folded according to an omega-shaped configuration and including:
a base divided into two sections located on opposite sides with respect to a central plane perpendicular to the base,
two intermediate flaps parallel to the base and located on opposite sides of said central plane, wherein the intermediate flaps are connected to respective ends of the base by respective first folds and are permanently fixed to the base,
two distal flaps parallel to the respective intermediate flaps and located on opposite sides of said central plane, wherein the distal flaps are connected to respective ends of the respective intermediate flaps by respective second folds and are separably fixed to the respective intermediate flaps, wherein said distal flaps protrude laterally with respect to the base beyond the respective first folds,
wherein the closing element comprises at least two surface fastening elements fixed on surfaces of said distal flaps opposite to the base,
wherein the base has a dimension in a direction perpendicular to the central plane equal to a distance between the first folds and comprised between 15 and 90 mm, and
wherein said intermediate flaps have a dimension in a direction perpendicular to the central plane equal to a distance between a first fold of the respective first folds and a second fold of the respective second folds, and comprised between 5 and 30 mm.

2. The closing element of claim 1, wherein a dimension in a direction perpendicular to the central plane equal to a distance between the second folds is comprised between 5 and 30 mm.

3. The closing element of claim 2, wherein a distance between each second fold and the central plane is less than a dimension of each intermediate flap.

4. The closing element of claim 1, wherein each distal flap has a dimension in a direction perpendicular to the central plane equal to a distance between the second fold and a respective end of the respective distal flap comprised between 10 and 76 mm.

5. The closing element of claim 4, wherein the dimension of each distal flap is greater than a sum of the dimension of each intermediate flap and half the dimension of the base.

6. The closing element of claim 1, wherein at least one surface fastening element of said at least two surface fastening elements has a dimension in a direction perpendicular to the central plane comprised between 5 and 25 mm.

7. The closing element of claim 6, wherein in each distal flap, a dimension in a direction perpendicular to the central plane equal to the distance between an end and an outer edge of a corresponding surface fastening element of said at least two surface fastening elements is comprised between 3 and 12 mm.
